(19) United States
(12) Patent Application Publication
(10) Pub. No.: US 2006/0009741 A1
(43) Pub. Date: Jan. 12, 2006

(54) Sealed medicine dropper containing a liquid

(76) Inventor: Jenn-Hae Luo, Taipei (TW)

Correspondence Address: LEONG C LEI, PMB # 1008, 1867 YGNACIO VALLEY ROAD, WALNUT CREEK, CA, 94598, US

(21) Appl. No.: 11/100,448

(22) Filed: Apr. 7, 2005

(30) Foreign Application Priority Data
Jul. 8, 2004 (TW) 093120413

(51) Int. Cl. A61M 35/00

(52) U.S. Cl. 604/310

(57) ABSTRACT

A sealed medicine dropper is disclosed. The dropper comprises a tube opening having a mouth section with a smaller diameter and the mouth section extended upwardly to pivotally mount with the tube body; the tube body being an extension from the tube opening with a larger tube diameter so as to fill a liquid; and a head end being a ball-like body at the rear end of the tube body and having a wall thickness smaller than that of the tube body facilitating depressing action of the fingers.

### BACKGROUND OF THE INVENTION

(a) Technical Field of the Invention

The present invention relates to a dropper, and in particular, a medicine dropper for containing a liquid medicine for children or old patients. The sealed dropper is disposed after use.

(b) Description of the Prior Art

Conventionally, medicines are placed on a spoon from a container or onto a small cup with scale measurement. However, the accuracy of the medicine is rather difficult to control, and the pouring of medicine onto a spoon is laborious. As shown in FIG. 1, a dropper 10′ is used and the medicine is enclosed within the dropper 10′. However, after one application, the dropper 10′ has to be cleaned for next application. In addition, the bottle containing medicine will not be used within a specific period. If the medicine is to be kept for further application, the bottle must be sealed. The drawback of the conventional dropper is that the whole bottle of medicine has to be carry along which is not convenient and there is a possible of misplaced the medicine. Accordingly, it is an object of the present invention to provide a medicine dropper which is sealed, which mitigates the above drawback.

### SUMMARY OF THE INVENTION

The primary purpose of the present invention is to provide a sealed medicine dropper having a tube body containing a liquid comprising a tube opening having a mouth section with a smaller diameter and the mouth section extended upwardly to pivotally mount with the tube body; the tube body being an extension from the tube opening with a larger tube diameter so as to fill a liquid; and a head end being a ball-like body at the rear end of the tube body and having a wall thickness smaller than that of the tube body facilitating depressing action of the fingers.

Yet another object of the present invention is to provide a sealed medicine dropper containing a liquid, wherein the mouth section of the tube opening is sealed with a foil.

Sill another object of the present invention is to provide a sealed medicine dropper containing a liquid, wherein the mouth section of the tube opening is provided with a score mark facilitating breakage of the tube opening.

A further object of the present invention is to provide a sealed medicine dropper containing a liquid, wherein the mouth section of the tube opening is threaded and is capped with a cap having inner threads, so as to seal the tube opening.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of a conventional medicine dropper.

FIG. 2 is a perspective view of a preferred embodiment of the present invention.

FIG. 3A is a sectional view of the first preferred embodiment of the present invention.

FIG. 3B is a sectional view of the first preferred embodiment of the present invention.

FIG. 4A is a sectional view of a second preferred embodiment of the present invention.

FIG. 4B is a sectional view of a second preferred embodiment, showing the application of the present invention.

FIG. 5A is a sectional view of a third preferred embodiment of the present invention.

FIG. 5B is a schematic view showing the sectional view of the third preferred embodiment.

FIG. 6A is a sectional view of the forth preferred embodiment of the present invention.

FIG. 6B is a sectional view of the forth preferred embodiment of the present invention.

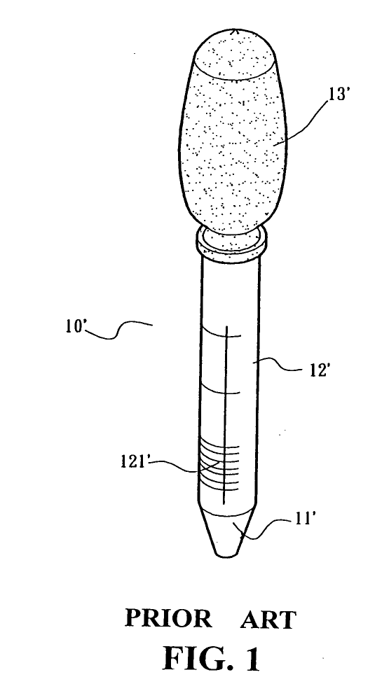

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

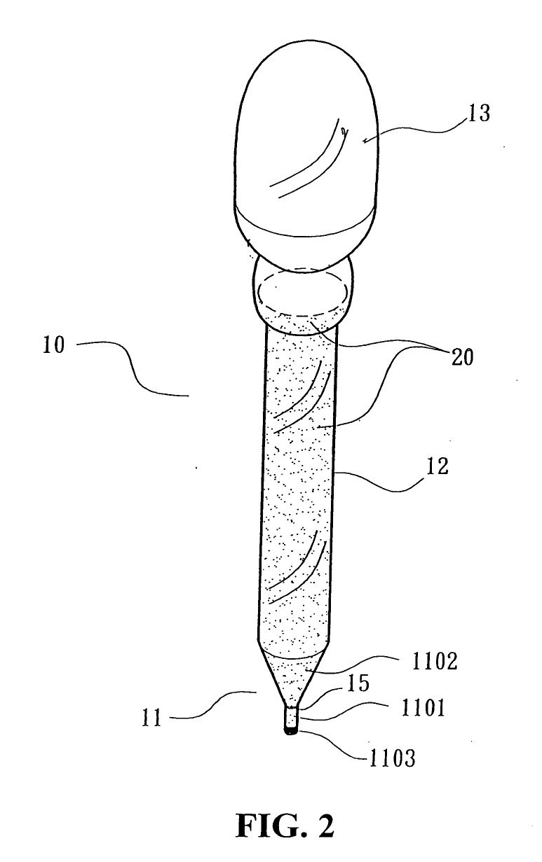

Referring to FIG. 2, there is shown a dropper 10 comprising a tube opening 11 having a mouth section 1101 with a smaller diameter, and the mouth section 1101 mounted with an extension 1102 so as to pivotally mouth to the tube body 12. The tube body 12 is an extension from the tube opening 11 and has a larger diameter to hold a liquid 20. The dropper 10 has a head end 13 is a ball-like body at the rear end of the tube body, and has a wall 1301 thickness thinner than the tube body. The tube opening 11, the tube body 12 and the head end 13 are formed as one unit, and the dropper 10 is filled with a liquid 20 (the head end 13 is provided with air) and is the tube opening 11 is sealed.

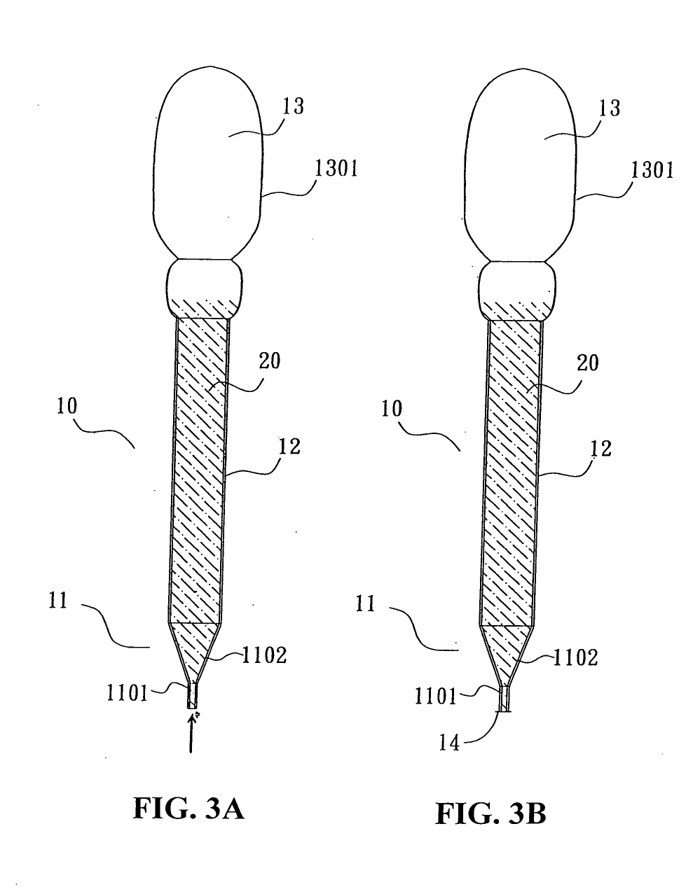

FIG. 3A is a first preferred embodiment of the present invention. Before a liquid 20 is filled into the tube opening 11 of the dropper 10, an appropriate amount of the liquid 20 is injected via the tube opening 11. As shown in FIG. 3B, a foil 14 is glued at the mouth section 1101 of the tube opening 11. Then the mouth section 1101 is sealed. When application, the foil 14 is torn off and the atmospheric pressure will prevent liquid 20 from flowing out. When the user presses the head end 13, the liquid 20 will flow out, and the pressure of the fingers controls the flow of the medicine.

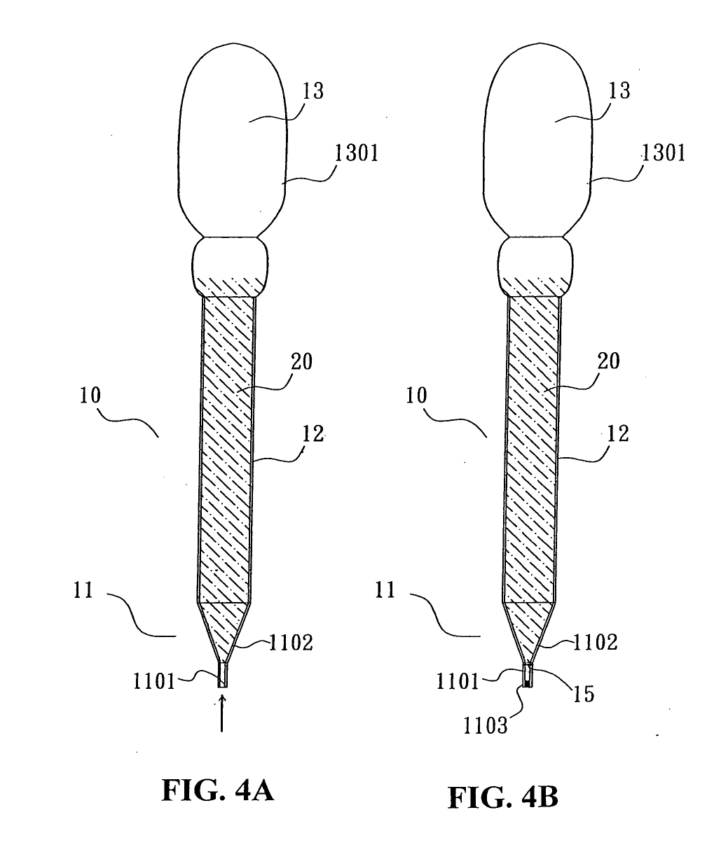

Referring to FIG. 4A, which shows a second preferred embodiment. The tube opening 11 is filled with a liquid 20 via them and a plug 1103 is inserted to the tube opening. The mouth section 1101 of the tube opening 11 is cut circular to form score mark 15. When the user tears off the score mark 15, the extension section 1102 will form an opening. Due to the atmospheric pressure, the liquid 20 will not flow out only when the user presses the head end 13.

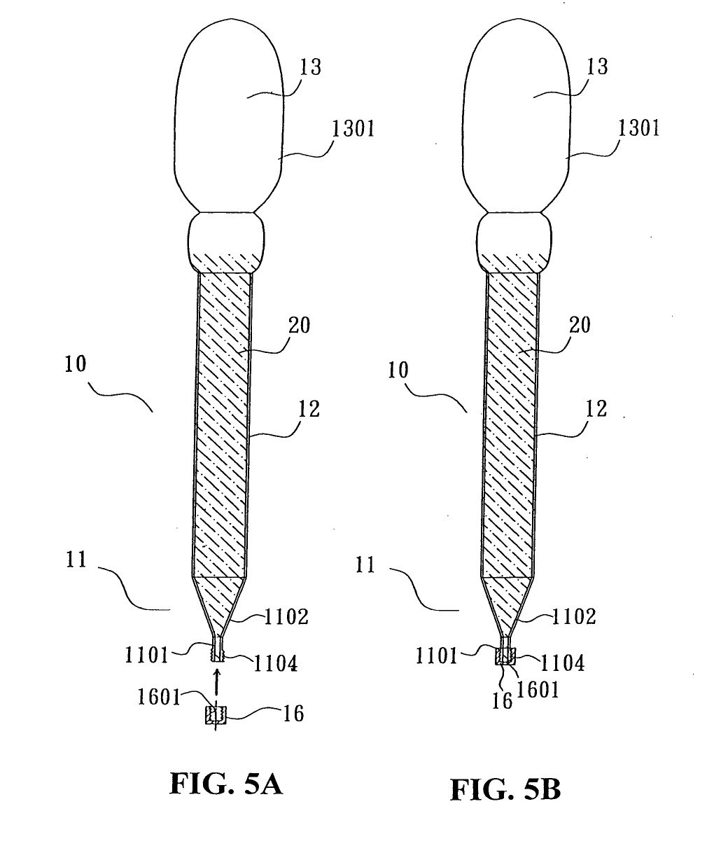

Referring to FIG. 5A showing a third preferred embodiment of the present invention. External threads 1104 are provided externally at the tube opening 11 and the tube opening 11 is filled with a liquid 20. As shown in FIG. 5B, a cap 16 with inner threads 1601 is mounted at the external threads of the tube opening 11 such that the cap 16 seals the tube opening. When in application, the cap 16 is unscrewed and the mouth section 1101 becomes an opening. The depression at the head end 13 will cause the liquid 20 to flow out.

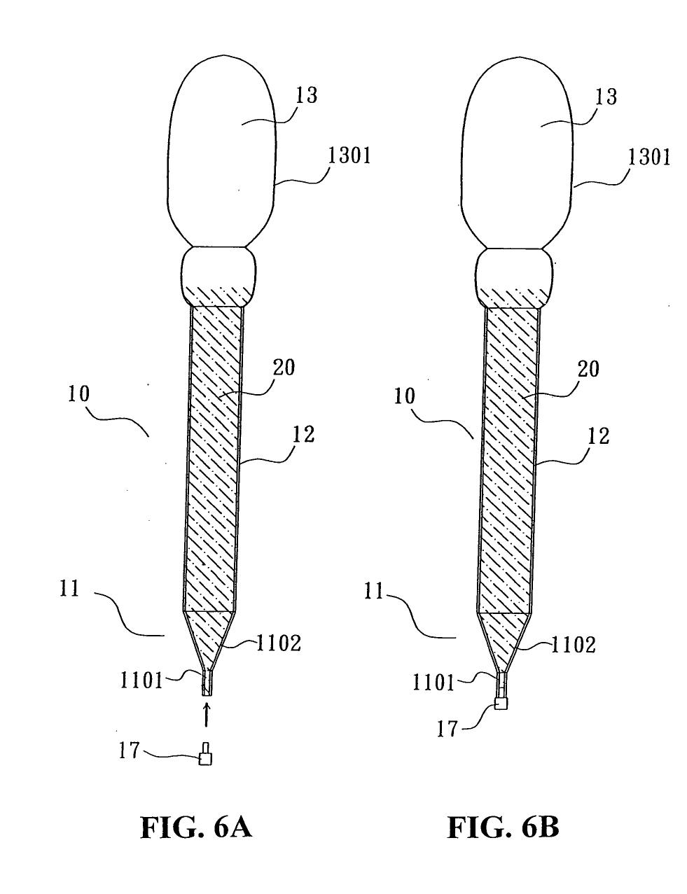

FIG. 6A shows a forth preferred embodiment. Before a liquid 20 is filled into the tube opening 11, as shown in FIG. 6B, a plug 17 of the protruded cross-section is inserted to the tube opening 11 and the plug seals the tube opening 11. When in application, the plug 17 is unplugged and the mouth section 1101 becomes an opening. At this instance, the atmospheric pressure prevents the liquid 20 of the dropper 10 from flowing out. When the user depresses the head end 13, the liquid 20 will flow out. The depression force controls the flow rate and amount of the liquid 20.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A sealed medicine dropper having a tube body containing a liquid comprising
a tube opening having a mouth section with a smaller diameter and the mouth section extended upwardly to pivotally mount with the tube body;
the tube body being an extension from the tube opening with a larger tube diameter so as to fill a liquid; and a head end being a ball-like body at the rear end of the tube body and having a wall thickness smaller than that of the tube body facilitating depressing action of the fingers.

2. The sealed medicine dropper of claim 1, wherein the mouth section of the tube opening is sealed with a foil.

3. The sealed medicine dropper of claim 1, wherein the mouth section of the tube opening is provided with a score mark facilitating breakage of the tube opening.

4. The sealed medicine dropper of claim 1, wherein the mouth section of the tube opening is threaded and is capped with a cap having inner threads, so as to seal the tube opening.

5. The sealed medicine dropper of claim 1, wherein a plug with a protruded cross-section is engaged with the tube opening so as to seal the tube opening.
